(19) United States
(12) Patent Application Publication
(10) Pub. No.: US 2003/0105531 A1
(43) Pub. Date: Jun. 5, 2003

(54) Multi-axial prosthetic ankle joint

(76) Inventor: Dwight Bunn, Poulsbo, WA (US)

Correspondence Address: Dwight Bunn, 18985 NE MEADOW RUN DRIVE, POULSBO, WA, 98370, US

(21) Appl. No.: 10/002,899

(22) Filed: Dec. 5, 2001

(51) Int. Cl. A61F 2/64; A61F 2/66

(52) U.S. Cl. 623/47; 623/48

(57) ABSTRACT

A multi-axial prosthetic ankle joint of the type having a modified Curtis Universal Joint part number CJ 647 (4); with a Century Spring (3) 1.625 inches modified to 2.562 inches in length, and a wire diameter of 0.243 inches superimposed; over which assembly a steel reinforced hose (8) 1.5 inches in diameter is placed.

### CROSS-REFERENCE TO RELATED APPLICATIONS

Not applicable

### BACKGROUND

1. Field of Invention

This invention relates to the field of human limb prosthetic devices, specifically lower extremity endo-skeletal systems.

2. Description of Prior Art

Most lower extremity endo-skeletal prosthetic systems do not have a provision for plantar extension and flexion of the foot. The feet are usually rigidly fixed to the system and any imitation of normal ankle motion is either provided by a semi-flexible mount in the foot or simply the flexibility of the materials used in the construction of the prosthetic foot. Since true ankle motion is lost, smooth normal gait is difficult for the amputee to achieve and more importantly rough or uneven terrain cannot be easily traversed. An amputee may lose balance and fall due to non-compliance of the foot to terrain changes. Kneeling by flexion of the ankle is impossible.

Attempts to create a flexible ankle system have largely been unsuccessful, and are usually incorporated into the design of the foot. The College Park Tru-Step™ foot can provide some limited flexibility as can several of the Flex-Foot™ systems, but there is provision for range of motion control with respect to the Anterior-Posterior and Medial-Lateral planes. Since a limited number of foot designs provide for this movement, an amputee must use one of these foot options if this function is to be incorporated in a prosthesis.

All of the ankle joint systems heretofore known suffer from a number of disadvantages:

(a) They do not allow for normal range of motion control in the Anterior-Posterior and Medial-Lateral planes, including flexion and extension.

(b) They do not allow for a kneeling posture by means of full ankle flexion of the prosthetic foot.

(c) They cannot be provided as a separate individual component of the prosthetic system, independent of foot system choice.

### SUMMARY

In accordance with the present invention a multi-axial prosthetic ankle joint comprises a modified Curtis Universal Joint part number CJ 647; with a spring superimposed (Century Spring Corporation) 1.625 inches by 2.563 inches, wire diameter 0.243 inches; over which a steel reinforced hose 1.5 inches in diameter is placed.

### OBJECTS AND ADVANTAGES

Accordingly, besides the objects and advantages of the multi-axial prosthetic ankle joint described in the above patent, several objects and advantages of the present patent are:

(a) to provide normal range of motion of the ankle in both the Anterior-Posterior and Medial-Lateral planes, including full flexion and extension.

(b) to provide for normal kneeling posture on the prosthetic side by means of full flexion of the prosthetic foot.

(c) to provide a separate component that could be incorporated into any endo-skeletal prosthetic system and could be used with any foot.

Further objects and advantages will become evident from a consideration of the following description and drawings.

### DRAWING FIGURES

In the drawings, FIGS. 1 and 3 have the same numerals

FIG. 1 shows the multi-axial prosthetic ankle joint in lateral aspect.

FIGS. 2A and 2B shows the individual components and modifications operations performed on both the both the spring and universal joint.

FIG. 3 shows the multi-axial prosthetic ankle joint in lateral aspect, right foot application in an endo-skeletal prosthetic system.

### REFERENCE NUMERALS IN DRAWINGS

1 allen head cap screw, 10 mm by 1.5 mm by 2 in long

2 star washer, 10 mm

3 spring, 1.625 in by 2.563 in, 0.243 in wire diameter

4 universal joint, Curtis Universal Joint CJ 647

5 setscrew, 10 mm by 1.5 mm, 0.750 in long

6 roll pin, 0.250 in by 1.500 in long

7 pylon tube, prosthetic endo-skeletal

8 reinforced steel hose

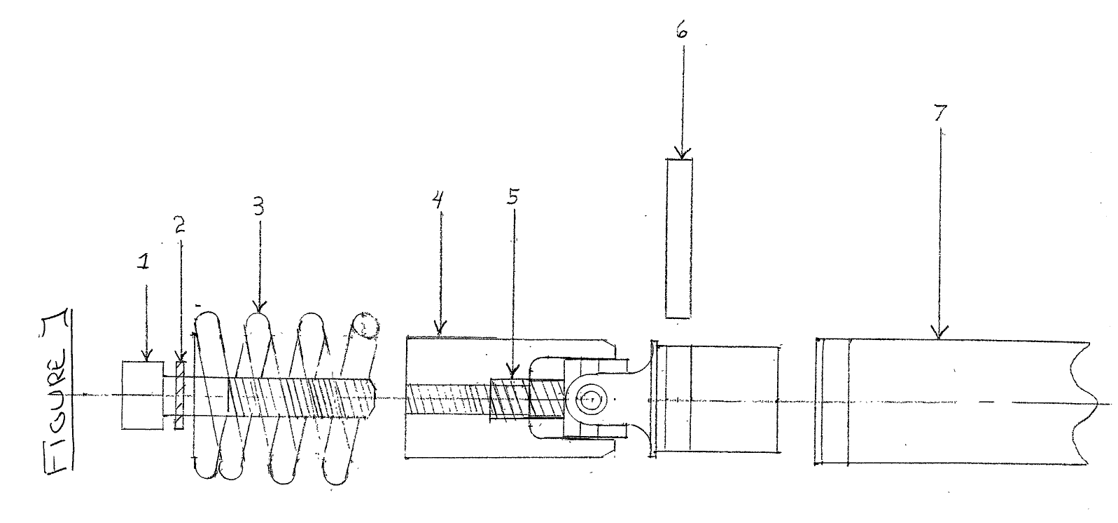

### DESCRIPTION—FIGS. 1, 2A AND 2B, AND 3

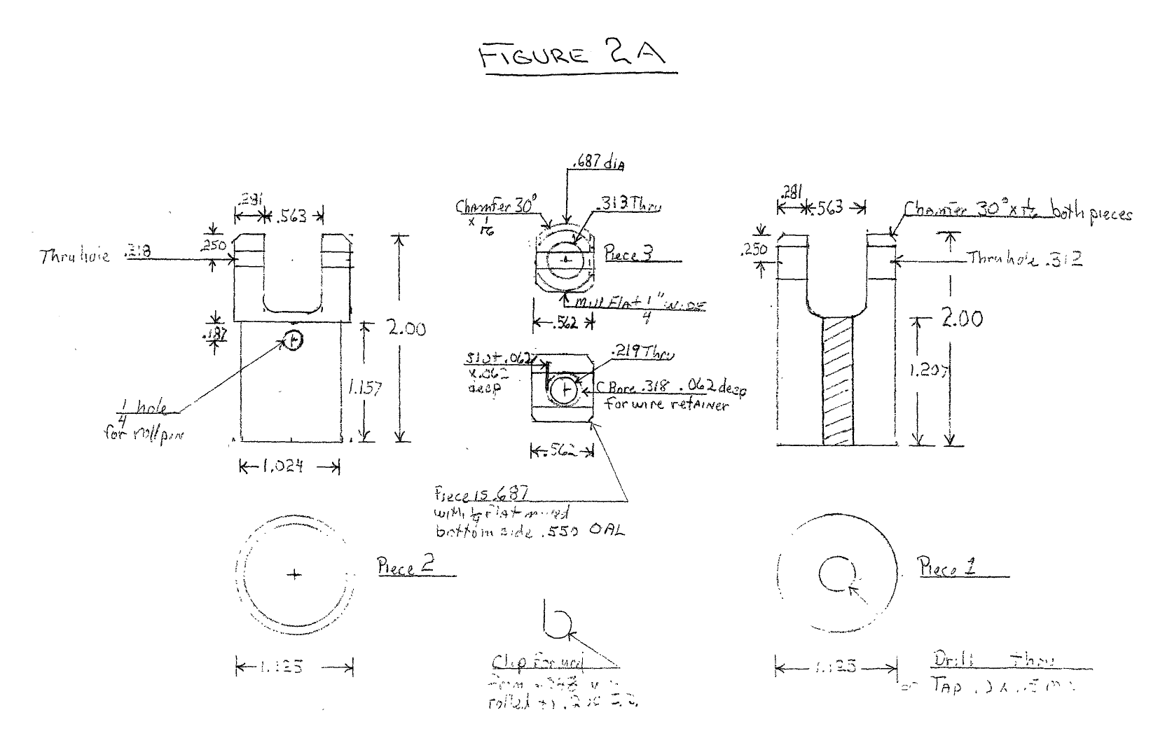

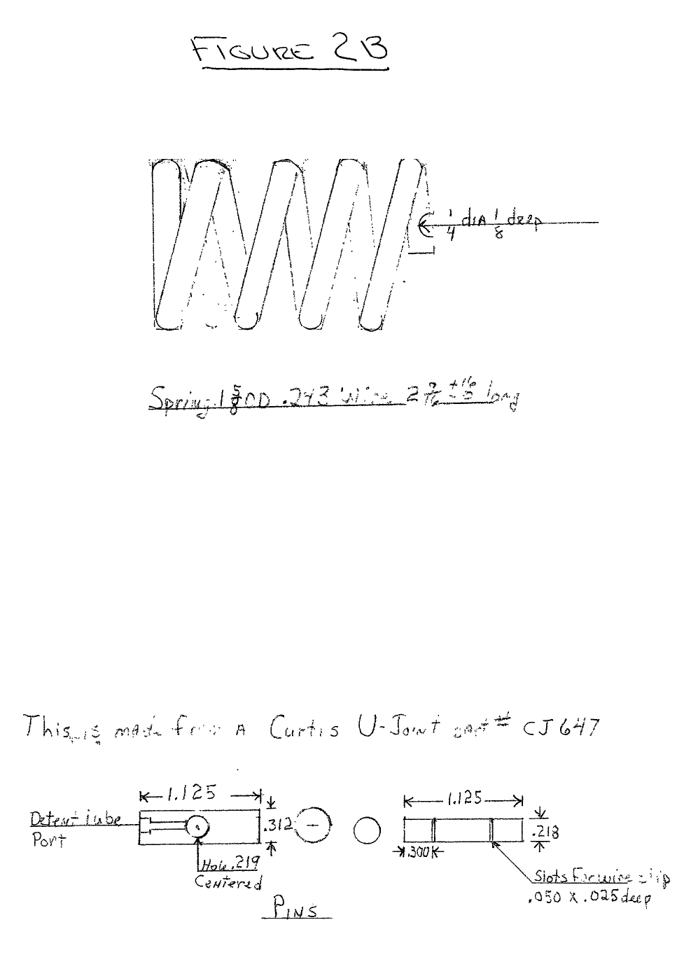

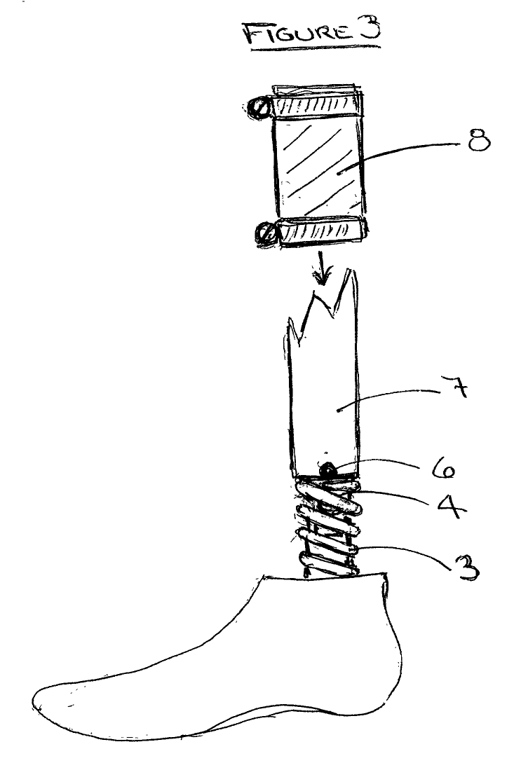

A preferred embodiment of the multi-axial prosthetic ankle joint of the present invention is illustrated in FIG. 1 (lateral aspect), FIGS. 2A and 2B (individual components), and FIG. 3 (in situ in a prosthetic system). Several steps are involved in constructing the multi-axial joint. A Curtis Universal Joint # CJ647 (4) provides the basis for the device. A land is machined in the end of the joint that has the large pin. It is dimensioned to 1.024 in by 1.157 inches long. The joint is then disassembled by removing the small center pin and retainer clip. The un-machined joint end is center drilled through its length 0.332 inches and tapped for 10 mm by 1.5 mm thread. The end of the joint previously machined can be inserted into an endo-skeletal prosthetic pylon tube (7), and both the tube and joint end cross-drilled to accept a 0.250 in by 1.5 in roll pin (6).

A Century Spring (3) 1.625 in outside diameter by 4.50 in long with a wire diameter of 0.243 in, is modified by cutting the length to 2.562 in long. On the cut end of the spring grind a 0.250 in slot 0.375 in from the end to aid assembly by providing slight spring compression. During assembly align the large pin with to joint oil port with the medial/lateral plane of the foot to restrict joint movement to 45 degrees in the anterior/posterior plane. Before assembly to the prosthetic foot, insert a 10 mm by 1.5 mm by 0.750 mm setscrew (5) into the center-drilled hole on the un-machined joint end. The screw is threaded up from the bottom against the center portion of the joint to prevent side-to-side rotation.

Alternative Embodiment

To damp the action of the spring (3) and provide a more natural gait, a piece of steel reinforced rubber hose (8) may be slipped over the assembled joint and clamped in place with worm drive hose clamps.

Advantages

From the description above, a number of advantages of the multi-axial prosthetic ankle joint become evident:

(a) The ankle joint permits a normal range of motion in both the Anterior-Posterior and Medial-Lateral planes, including full foot flexion and extension.

(b) Normal anatomical kneeling posture may be assumed with the prosthetic limb by means of full flexion of the prosthetic foot through weight bearing.

c) The multi-axial ankle joint can be provided as an individual component of any endo-skeletal prosthetic system independent of prosthetic foot or residual limb interface system.

Operation—FIGS. 1 and 3

The manner of installing and using the multi-axial prosthetic ankle joint is as follows: Slide the spring (3) on over the joint from the distal end. The spring (3) will register in the 0.250 in ground slot, and will be slightly longer than the universal joint (4). Assembly the 10 mm star washer (2) over the 10 mm by 1.5 mm allen head cap screw (1) and insert through the prosthetic foot assembly and tighten. If a different foot system is selected an appropriate length allen head cap screw (1) may be employed to supply spring compression and joint mounting. The joint will extend and flex as the prosthesis user bears weight through the gait cycle or when kneeling.

### CONCLUSIONS, RAMIFICATION, AND SCOPE

Accordingly, the reviewer will see that the multi-axial prosthetic ankle joint of this invention can supply increased function and more natural anatomical movement. Furthermore, it has the additional advantages in that:

(a) it permits an anatomically normal range of motion in both the Anterior-Posterior and Medial-Lateral planes, including full foot extension and flexion.

(b) it allows for anatomically normal kneeling posture on the prosthetic limb by means of full flexion of the ankle.

(c) it can be provided as an individual component of any lower extremity endo-skeletal prosthetic limb system, independent of prosthetic foot or residual limb interface system choices.

Although the description above contains many specificities, these should not be construed as limiting the scope of the invention, but merely as providing examples of the presently preferred embodiments of this invention. For example, various spring configurations could be employed, including variable compression rate designs to alter the return response depending on flexion or extension angle of the prosthetic foot.

Thus the scope of the invention should be determined by the appended claims and their legal equivalents, rather than by the example given.

I claim:

1. In a multi-axial prosthetic ankle joint of the type having a modified Curtis Universal Joint, over which a modified Century Spring is superimposed and over which assembly a steel reinforced hose is placed.

2. In the ankle joint of claim 1 wherein said joint is a Curtis Universal Joint part number CJ 647.

3. The ankle joint of claim 2 wherein said joint is modified by machining a land 1.024 in by 1.157 in long on the large pin end, and center drilling a 0.332 hole through the un-machined end of the joint and tapping to 10 mm by 1.5 mm thread.

4. The ankle joint of claim 1 wherein said Century Spring has and outside diameter of 1.625 in by 4.50 inches in length with a wire diameter of 0.243 in.

5. The ankle joint of claim 4 wherein said spring is modified by cutting the length to 2.562 in and on the cut end grinding a 0.250 slot 0.375 in from the cut end.

6. The ankle joint of claim 1 wherein said joint assembly is covered by a steel reinforced flexible rubber hose, 1.5 in inside diameter.
